(19) United States
(12) Patent Application Publication
(10) Pub. No.: US 2020/0269073 A1
(43) Pub. Date: Aug. 27, 2020

(54) EXTRACORPOREAL SHOCK WAVE ULTRASOUND FOR ENHANCEMENT OF REGENERATIVE ACTIVITIES IN ERECTILE DYSFUNCTION

(71) Applicant: CREATIVE MEDICAL TECHNOLOGIES, INC., Phoenix, AZ (US)

(72) Inventors: Tim WARBINGTON, Phoenix, AZ (US); Amit PATEL, Salt Lake City, UT (US); Thomas ICHIM, San Diego, CA (US)

(73) Assignee: CREATIVE MEDICAL TECHNOLOGIES, INC., Phoenix, AZ (US)

(21) Appl. No.: 16/799,656

(22) Filed: Feb. 24, 2020

Related U.S. Application Data
(60) Provisional application No. 62/809,288, filed on Feb. 22, 2019.

(51) Int. Cl. A61N 7/00; A61K 35/28

(52) U.S. Cl. CPC A61N 7/00; A61N 2007/0034; A61K 35/28

(57) ABSTRACT

Disclosed are methods useful for the treatment of erectile dysfunction through stimulation of regenerative activities in a patient in need of treatment. In one embodiment, the invention provides the administration of extracorporeal shock waves prior to administration of regenerative cells in order to enhance the penile microenvironment in a manner favorable for augmented regenerative activity. In some embodiments said regenerative cells comprise of autologous bone marrow cells. In other embodiments regenerative cells are allogeneic cells. The invention further teaches the use of regenerative cell mobilization combined with administration of extracorporeal shock wave therapy.

### CROSS-REFERENCE TO RELATED APPLICATIONS

This application claims the benefit of priority to U.S. Provisional Application No. 62/809,288, filed Feb. 22, 2019, which is incorporated herein by reference.

### BACKGROUND OF THE INVENTION

Erectile dysfunction has gained widespread attention and acceptance within the past decades as a physical disorder. The lack of either generating or keeping an erection has created problems in certain portions of the male population as sexual activity generally requires the ability to provide effective penetration; without such an erect member. Although such problems have existed historically, recent advancements have attempted to overcome these deficiencies. Actual implants, with, for instance, external electrical stimulation, have been utilized in the past, though these have never proven to be of great effectiveness, let alone, desirable to any real extent. Chemical remedies have thus been investigated with a number showing highly effective results. Such developments, including phosphodiesterase type 5 (PDE-5) inhibitors like sildenafil citrate (VIAGRA), tadalafil (CIALIS), and the like, function through oral delivery and subsequent in vivo generation of nitric oxide at or near the penis thereby causing blood to enter the sinuses therein to create the necessary erection.

Unfortunately, in about 30% of patients the mechanical means associated with generation erections are damaged and thus not responsive to nitric oxide, therefore making the use of PDE-5 inhibitors useless. Therapies such as cellular interventions have utilized in animal models, as well as in clinical trials, these include skeletal muscle cells [1]. Additionally, gene therapy has been utilized in some preclinical and early clinical models [2-6]. In some studies transfected genes included eNOS [7-15], ion channels [16-21], prepro-calcitonin peptide [22], VEGF [23-28], BDNF [29, 30], vasoactive intestinal peptide [31], neurotrophin-3 [32], angiopoietin-1 [33], superoxide dismutase [34], IGF-1 [35], suppression of protein inhibitor of NOS [36], GDNF [37], neurturin [38], telomerase reverse transcriptase [39], hypoxia inducible factor [40], inhibition of IGF-binding protein 3 [41, 42], myocardin [43], inhibition of ninjurin-1 [44], inhibition of ROCK2 [45], human tissue kallikrein-1 [46],

For both stem cells and gene therapy, clinical results to date support safety and some signals of efficacy [47-49].

Unfortunately, not all patients are responsive and novel means of augmenting efficacy of regeneration are needed. The current invention teaches means of using extracorporeal shock wave therapy for augmentation of regenerative approaches.

### SUMMARY

Certain aspects are directed to a method for the stimulation of penile regeneration, the method comprising: a) associating the corpus cavernosum of a patient in need of therapy with a shockwave generating device; b) applying a shockwave regimen to said corpus cavernosum wherein said shockwave device produces a focal zone comprising at least a portion of the corpus cavernosum; and c) administering a regenerative cell population.

Certain aspects are directed to methods wherein said association of said corpus cavernosum with said shockwave device is performed in an aqueous environment.

Certain aspects are directed to methods wherein said focal zone includes areas of the corpus cavernosum which possess reduced circulation, and/or degenerative features.

Certain aspects are directed to methods wherein said shockwave regimen locally promotes at least one or more biological activities chosen from the group consisting of: a) angiogenesis; b) enhanced perfusion of the penis, c) mitogenesis of smooth muscle cells; and d) augmentation of endothelial function.

Certain aspects are directed to methods wherein said shock wave regimen produce a treatment regimen determined based on at least one parameter chosen from the group consisting of shockwave parameters, treatment protocol parameters, and anatomical parameters.

Certain aspects are directed to methods wherein said shockwave parameters comprise number of shockwaves, frequency of shockwaves and intensity of said shockwave.

Certain aspects are directed to methods wherein said parameters are selected from of at least one of: shockwave intensity is about from about 50 bar to about 200 bar; shockwave frequency is from about 60 to about 300 shockwaves per min; said number of shockwaves is up to about 3500 per session.

Certain aspects are directed to methods wherein said anatomical parameters dividing the corpus cavernosum into treatment zones.

Certain aspects are directed to methods wherein said anatomical parameters comprise a single focal zone including up to about 90% of one or more areas identified as being subject to degenerative changes.

Certain aspects are directed to methods further comprising coupling said shock wave regimen with a drug or cellular treatment.

Certain aspects are directed to methods wherein an additional therapeutic agent is administered into the corpus cavernosum.

Certain aspects are directed to methods wherein the additional therapeutic agent is selected from the group consisting of growth factors, differentiation factors, regenerative cells, and nutritional supplements.

Certain aspects are directed to methods wherein the additional therapeutic agent is a growth factor.

Certain aspects are directed to methods wherein the additional therapeutic agent and the cells are administered into the corpus cavernosum using a carrier.

Certain aspects are directed to methods wherein the carrier is selected from the group consisting of beads, microspheres, nanospheres, hydrogels, gels, polymers, ceramics, collagen and platelet gels.

Certain aspects are directed to methods wherein the additional therapeutic agent is administered simultaneously with administering the cells to the corpus cavernosum.

Certain aspects are directed to methods wherein the additional therapeutic agent is administered prior to administering the cells to the corpus cavernosum.

Certain aspects are directed to methods wherein the additional therapeutic agent is administered after administering the cells to the corpus cavernosum.

Certain aspects are directed to methods wherein the cells are administered into the corpus cavernosum in a formulation with a volume of between about 0.1 ml and about 2 ml.

Certain aspects are directed to methods wherein the carrier comprises a hydrogel.

Certain aspects are directed to methods wherein the carrier comprises micro spheres.

Certain aspects are directed to methods wherein the additional therapeutic agent is stromal derived factor 1.

Certain aspects are directed to methods wherein the therapeutic agent is platelet concentrate.

Certain aspects are directed to methods wherein the cells are administered into the corpus cavernosum.

Certain aspects are directed to methods wherein the cells are administered systemically.

Certain aspects are directed to methods wherein the cells are selected from the group consisting of: a) bone marrow mononuclear cells; b) mesenchymal stem cells; c) adipose derived stem cells; and d) Wharton's Jelly derived stem cells.

Certain aspects are directed to methods wherein said cells are treated with one or more factors capable of stimulating smooth muscle differentiation.

Certain aspects are directed to methods wherein said factors capable of stimulating smooth muscle differentiation are selected from a group comprising of: a) IL-10; b) IL-20; c) IL-25; d) GDF-5; e) GDF-11; f) BMP-13; g) MIA/CD-RAP; h) PDGF-BB; i) FGF; j) IGF and k) dexamethasone.

Certain aspects are directed to methods wherein said regenerative cells are CD56 expressing mesenchymal stem cells.

Certain aspects are directed to methods wherein said CD56 mesenchymal stem cells are derived from sources selected from a group comprising of: a) foreskin; b) adipose tissue; c) skin biopsy; d) bone marrow; e) placenta; f) umbilical cord; g) placenta; h) umbilical cord blood; i) ear lobe skin; and j) embryonic fibroblasts.

Certain aspects are directed to methods wherein said mesenchymal are cultured under conditions of hypoxia at a sufficient lack of oxygen and for a sufficient time period to induce expression of HIF-1 alpha.

Certain aspects are directed to methods wherein expression of HIF-1 alpha is detected by expression of VEGF secretion.

Certain aspects are directed to methods wherein said hypoxia comprises incubation of said mesenchymal stem cells in a hypoxic environment from 0.1% oxygen to 10% oxygen for a period of 30 minutes to 3 days.

Certain aspects are directed to methods wherein said mesenchymal stem cells are cultured at 3% oxygen for 24 hours.

Certain aspects are directed to methods wherein said hypoxia induces upregulation of CXCR4 receptor on said mesenchymal stem cells cells.

Certain aspects are directed to methods wherein said upregulation of said CXCR4 endows said mesenchymal stem cells with upregulated ability to home to an SDF-1 gradient.

Certain aspects are directed to methods wherein said hypoxia is induced by chemical means.

Certain aspects are directed to methods wherein said chemical means of induction of hypoxia comprise of culture in cobalt (II) chloride.

Certain aspects are directed to methods wherein said mesenchymal stem cells are incubated with 1 uM-300 uM cobalt (II) chloride for a time period of 1-48 hours.

Certain aspects are directed to methods wherein said mesenchymal stem cells are incubated with 250 uM of cobalt (II) chloride for a time period of 24 hours.

Certain aspects are directed to methods wherein said mesenchymal stem cells are treated in a manner so as to suppress expression of apoptosis associated genes.

Certain aspects are directed to methods wherein said treatment is exposure to an antisense oligonucleotide capable of inhibiting said apoptosis associated genes.

Certain aspects are directed to methods wherein said antisense oligonucleotide is capable of activating RNAse H targeting said apoptosis associated gene.

Certain aspects are directed to methods wherein said suppression of apoptosis associated genes is achieved by administration of an agent capable of inducing RNA interference.

Certain aspects are directed to methods wherein said agent capable of inducing RNA interference is short interfering RNA.

Certain aspects are directed to methods wherein said agent capable of inducing RNA interference is short hairpin RNA.

Certain aspects are directed to methods wherein said apoptosis associated genes are selected from a group comprising of: Fas, FasL, CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, and TRAF4.

Certain aspects are directed to methods of treating erectile dysfunction comprising the steps of: a) administering ultrasound shock waves; and b) administered a transplanted cellular population.

Certain aspects are directed to methods wherein said transplanted cell is a bone marrow mononuclear cell population.

Certain aspects are directed to methods wherein said bone marrow mononuclear cell population is purified for CD133 positive cells.

Certain aspects are directed to methods wherein said cell population is adipose stromal vascular fraction cells.

Certain aspects are directed to methods wherein said adipose stromal vascular fraction cells are used as a source of mesenchymal stem cells.

Certain aspects are directed to methods wherein said mesenchymal stem cells are autologous or allogeneic.

Certain aspects are directed to methods wherein said bone marrow cells are isolated based on expression of CD34.

Certain aspects are directed to methods wherein said bone marrow cells are isolated based on expression of aldehyde dehydrogenase activity.

Certain aspects are directed to methods wherein said cells are mobilized peripheral blood stem cells comprised of a heterogeneous leukopheresis product extracted from a donor after mobilization.

Certain aspects are directed to methods wherein said mobilized peripheral blood stem cells are comprised of an isolated CD34 cell derived from heterogeneous leukopheresis product extracted from a donor after mobilization.

Certain aspects are directed to methods wherein said cells are cord blood derived stem cells.

Certain aspects are directed to methods wherein said cord blood derived stem cells comprise a population selected for expression of CD34.

Certain aspects are directed to methods wherein said cord blood derived stem cells comprise a population selected for expression of CD133.

Certain aspects are directed to methods of treating suboptimal function of corpus cavernosal tissue comprising the steps of: a) selecting a patient whose corpus cavernosal tissue is functioning suboptimally; b) providing said patient a mobilizing agent or therapy sufficient in increase circulating regenerative cells; and c) providing said patient a chemoattractant means to selectively induce homing of said regenerative cells to said corpus cavernosal tissue.

Certain aspects are directed to methods wherein said regenerative cells are autologous bone marrow cells.

Certain aspects are directed to methods wherein said augmentation of regenerative cell numbers in circulation is performed by administration of regenerative cells into a patient.

Certain aspects are directed to methods wherein said mobilization is accomplished by administration of G-CSF.

Certain aspects are directed to methods wherein said chemoattractant means comprises administration of an extracorporeal shock wave.

Certain aspects are directed to methods wherein said extracorporeal shockwave is administered using a device, wherein said device is in contact with said corpus cavernosum.

Certain aspects are directed to methods wherein parameters for said shock wave are selected from of at least one of: shockwave intensity is about from about 50 bar to about 200 bar; shockwave frequency is from about 60 to about 300 shockwaves per min; said number of shockwaves is up to about 3500 per session.

Certain aspects are directed to methods wherein an additional therapeutic agent is administered into the corpus cavernosum.

Certain aspects are directed to methods wherein the additional therapeutic agent is selected from the group consisting of growth factors, differentiation factors, regenerative cells, and nutritional supplements.

Certain aspects are directed to methods wherein the additional therapeutic agent is a growth factor.

Certain aspects are directed to methods wherein the additional therapeutic agent and the cells are administered into the corpus cavernosum using a carrier.

Certain aspects are directed to methods wherein the carrier is selected from the group consisting of beads, microspheres, nanospheres, hydrogels, gels, polymers, ceramics, collagen and platelet gels.

Certain aspects are directed to methods wherein the additional therapeutic agent is administered simultaneously with administering the cells to the corpus cavernosum.

Certain aspects are directed to methods wherein the additional therapeutic agent is administered prior to administering the cells to the corpus cavernosum.

Certain aspects are directed to methods wherein the additional therapeutic agent is administered after administering the cells to the corpus cavernosum.

### BRIEF DESCRIPTION OF THE DRAWINGS

FIG. 1 is a bar graph showing the results of extracorporeal shock wave on increasing bone marrow production of regenerative cytokines: IGF, VEGF, and PDGF.

### DETAILED DESCRIPTION OF THE INVENTION

The use of extracorporeal ultrasound is disclosed for “preactivating” stem cells, particularly bone marrow stem cells, for use in the treatment of erectile dysfunction. The invention provides means of “preactivating” a cellular graft before implantation. Said “preactivation” refers to induction of biochemical processes within the graft so as to allow for: a) increased viability; b) augmented function; c) accelerated integration with the tissue in which implantation of cellular graft has occurred. In one particular embodiment.

In the context of the present invention, the term “regenerative cell” refers to a mammalian stem cell, progenitor cell, or differentiated cell. As used herein, embryonic, fetal/placental and adult-derived cellular populations are included in the definition of “regenerative cells”. In the context of the present invention, regenerative cells may include: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated tooth derived stem cells, hair follicle stem cells, dermal stem cells, parthenogenically derived stem cells, reprogrammed stem cells and side population stem cells and the like.

As used herein, “stem cell” refers to a pluripotent cell capable of differentiating into numerous cellular lineages.

As used herein, the term “progenitor cell” refers to a lineage-committed cell that may have undergone various stages of differentiation toward a tissue-restricted cell type. The term “differentiated cell” used in the context of the present invention refers to a tissue-restricted cell.

As used herein, the term “mesenchymal stem cell” refers to a multipotent stem cell that can differentiate into a variety of cell types, including: osteoblasts (bone cells), chondrocytes (cartilage cells), and adipocytes (fat cells).

As used herein, “therapeutic energy treatment” refers to any light-, acoustic-, electrical-, or mechanical means of stimulating cells or tissue through means either applied externally to cells (i.e. regenerative cells) or directly to the tissue (specifically, the penis in the context of the present invention) for therapeutic purposes.

As used herein, “biological dysfunctions” used in the context of erectile dysfunction refers to the deficiencies or abnormalities in cellular numbers or functions that underlie the inability of an individual to attain an erection. Said biological dysfunctions are diagnosed in the art using established medical tests and diagnostic criteria. These include but are not limited to the following tests: Doppler Ultrasound, dynamic infusion cavernosometry & cevernosography, tests of penile nerve function including but not limited to the bulbocavernosus reflex test, nocturnal penile tumescence testing, penile biothesiometry, and corpus cavernosometry. Examples of biological dysfunctions present in ED include arteriogenic changes in blood vessels and loss of smooth muscle mass.

As used herein, a “therapeutically effective amount” and “therapeutically sufficient amount” and like terms refer to an amount of an agent sufficient to ameliorate at least one symptom, behavior or event, associated with a pathological, abnormal or otherwise undesirable condition, e.g., erectile dysfunction, or an amount sufficient to prevent or lessen the probability that such a condition will occur or re-occur, or an amount sufficient to delay worsening of such a condition. In one embodiment, the term therapeutically effective amount and like terms are used to refer to the frequencies and concentrations at which regenerative cells are administered for treating erectile dysfunction.

As used herein, “extracorporeal shockwave” used in the present disclosure refers to a continuous single sound wave generated by a specific sound generator, which has a high peak pressure amplitude of up to 100 MPa, has a short duration of less than 1 μs, and delivers to a specific target area with an energy density in the range of 0.005 mJ/mm.sup.2 to 1.0 mJ/mm.sup.2.

Extracorporeal shockwave therapy (herein referred to as ‘ESWT’) is non-surgical, non-invasive treatment of medical conditions using acoustic shockwaves. First use of shockwave therapy in the early 1980's was utilized to fragment kidney stones termed shockwave lithotripsy. Continued development of shockwave treatment showed the possibility of stimulating bone formation, angiogenesis, as well as other orthopedic indications. However, medical literature suggests that lithotripsy creates hypertension and some damage to the kidney including hematuria during the procedure. A shockwave is a form of acoustic energy resulting from phenomena that create a sudden intense change in pressure for example an explosion or lightning. The intense changes in pressure produce strong waves of energy that can travel through any elastic medium such as air, water, human soft tissue, or certain solid substances such as bone. Shockwaves are characterized by the delivery of a sequence of transient pressure disturbances characterized by an initial high peak pressure with a fast pressure rise followed by rapid wave propagation with diminishing amplitude over its lifecycle. Such that shockwaves characteristically have a quick lifecycle, starting with a big high amplitude pressure peak followed by a gradual diminishing pressure amplitude having amplitude of about 10-20% of the initial pressure peak. Shockwave are further characterized in that they do not produce heat within the tissue. Shockwaves are therefore characteristically different from ultrasound in that the ultrasound waveform produces constant cyclic sinusoidal amplitude that produces heat at the tissue level. Conversely shockwaves do not have constant amplitude over time. Acoustic shockwaves are primarily generated by three different methods, electrohydraulic (also referred to as spark gap), electromagnetic (also referred to as ‘EMSE’), and piezoelectric. Each method needs an apparatus to focus the generated shockwave so as to provide a focal point and/or focal zone for the treatment area. In the focal zone shockwaves produce much higher pressure impulses as compared with the zones outside of the focal zone. Mechanical means for focusing each of these methods is generally realized with an appropriate arrangement of surfaces reflecting the wave toward the desired focal point and/or an appropriate arrangement of the generating devices. Spark gap systems incorporate an electrode (spark plug), to initiate a shockwave, and ellipsoid to focus the shockwave. EMSE systems utilize an electromagnetic coil and an opposing metal membrane. Piezoelectric systems form acoustical waves by mounting piezoelectric crystals to a spherical surface to provide focus. Of the three systems, the spark gap system is generally preferred in the art for generating therapeutic shockwaves ESWT as it introduces more of the generated shockwave energy to the treatment target site. In spark gap systems, high energy shockwaves are generated when electricity is applied to an electrode positioned in an ellipsoid immersed in treated water. When the electrical charge is fired, a small amount of water is vaporized at the tip of the electrode and a shockwave is produced. The shockwave ricochets from the side of an ellipsoid and converges at a focal point, which may then be transferred to the area to be treated. In electromagnetic systems an electrical impulse is circulated in a coil. The coil produces an electromagnetic field that expels a metallic membrane to produce the mechanical impulse. In piezoelectric systems ceramic material with piezoelectric characteristics is subjected to an electrical impulse. The electric impulse modifies the dimension of the ceramic material to generate the desired mechanical impulse. A focal point is attained by covering a concave spherical surface with piezoelectric ceramics converging at the center of the sphere. The method of focusing the generated shockwave has been greatly described in the art for example in U.S. Pat. Nos. 5,174,280 and 5,058,569, 5,033,456, EP1591070 all of which are incorporated herein by reference as if fully set forth. Traditionally shockwaves have been used in medicine as a noninvasive means for treating a variety of anomalies such as kidney stones (lithotripsy), fragmentation of calcification, chronic orthopedic inflammation healing, bone healing (osteogenesis), wound healing, revascularization, angiogenesis are well known and described in medical literature. U.S. Pat. No. 7,507,213 to Schultheis s, et al. discusses invasive stimulation of kidney by surgically exposing the organ for example heart or kidney prior to applying shockwave therapy. US Patent Publication No. 2011/0257523 to Hastings et al. discusses a method utilizing high intensity focused ultrasound (HIFU) for ablating innervated tissue of the kidney, for denervating renal vasculature, including disruption and termination of renal sympathetic nerve activity, to improve cardiac and/or renal function particularly that associated with hypertension.

Cells can be mobilized by administration of a mobilizing agent or therapy. The mobilizing agent can be selected from a group that includes: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA-reductase inhibitors and small molecule antagonists of SDF-1 and the like. Further, the mobilization therapy can be selected from a group that includes: exercise, hyperbaric oxygen, autohemotherapy by ex vivo ozonation of peripheral blood, and induction of SDF-1 secretion outside of the bone marrow.

In certain aspects of the present invention, the committed hematopoietic progenitor cells can express the marker CD133 or CD34.

In specific embodiments of the invention, the regenerative cells are expanded in culture using one or more cytokines, chemokines and/or growth factors prior to administration to an individual in need thereof. The agent capable of inducing stem cell expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family. The agent capable of inducing stem cell differentiation can be selected from HGF, BDNF, VEGF, FGF1, FGF2, FGF4, and FGF 20.

For use in the present invention, said regenerative cells may be administered to an individual in need thereof via one or more of the following routes of administration: intravenously, intramuscularly, intraperitoneally, transdermally, or by any parenteral route. In a preferred embodiment of the present invention, said regenerative cells are administered intracavernosally.

Furthermore, conditions promoting certain type of cellular proliferation or differentiation can be used during the culture of said regenerative cells. These conditions include but are not limited to, alteration in temperature, alternation in oxygen/carbon dioxide content, alternations in turbidity of said media, or exposure to small molecules modifiers of cell cultures such as nutrients, inhibitors of certain enzymes, stimulators of certain enzymes, inhibitors of histone deacetylase activity such as valproic acid For use in the present invention, said regenerative cells may be administered to an individual in need thereof via one or more of the following routes of administration: intravenously, intramuscularly, intraperitoneally, transdermally, or by any parenteral route. In a preferred embodiment of the present invention, said regenerative cells are administered intracavernosally (i.e. directly into the corpus cavernosa of the penis).

In a preferred embodiment of the present invention, said regenerative cells are treated with a therapeutic energy ultrasound therapeutic energy treatment may be administered to regenerative cells in vitro and can be selected from one or more of the following: electrical stimulation of cells, and/or shockwave therapy of cells. Without being bound by theory, these therapeutic energy treatments can increase the therapeutic efficacy of the cellular product used to treat erectile dysfunction by altering one or more of the following activities of regenerative cells: proliferation, differentiation, survival, cytokine and chemokine production, and migration/homing to specific tissue sites.

It is known that inflammatory mediators augment the damaging effects of diabetes on erectile function. In one study, in vitro organ bath experiments were used to measure cavernosal reactivity in mice infused with vehicle or TNF-alpha (220 ng/kg/min) for 14 days. Gene expression of nitric oxide synthase isoforms was evaluated by real-time polymerase chain reaction. Corpora cavernosa from TNF-alpha-infused mice exhibited decreased nitric oxide (NO)-dependent relaxation, which was associated with decreased endothelial nitric oxide synthase (eNOS) and neuronal nitric oxide synthase (nNOS) cavernosal expression. Cavernosal strips from the TNF-alpha-infused mice displayed decreased nonadrenergic-noncholinergic (NANC)-induced relaxation (59.4+/−6.2 vs. control: 76.2+/−4.7; 16 Hz) compared with the control animals. These responses were associated with decreased gene expression of eNOS and nNOS (P<0.05). Sympathetic-mediated, as well as phenylephrine (PE)-induced, contractile responses (PE-induced contraction; 1.32+/−0.06 vs. control: 0.9+/−0.09, mN) were increased in cavernosal strips from TNF-alpha-infused mice. Additionally, infusion of TNF-alpha increased cavernosal responses to endothelin-1 and endothelin receptor A subtype (ET(A)) receptor expression (P<0.05) and slightly decreased tumor necrosis factor-alpha receptor 1 (TNFR1) expression (P=0.063). The conclusion was that corpora cavernosa from TNF-alpha-infused mice display increased contractile responses and decreased NANC nerve-mediated relaxation associated with decreased eNOS and nNOS gene expression. These changes may trigger ED and indicate that TNF-alpha plays a detrimental role in erectile function [50-52]. Accordingly, in one embodiment of the patent, inhibitors of TNF-alpha are administered along with stem cells and/or extracorporeal shock wave therapy.

In one embodiment of the invention, stem cells are administered alone, or in combination with mesenchymal stem cells into the corpus cavernosum to augment regeneration of the corpus cavernosum. Prior to, concurrent with, and/or subsequent to administration of cells, extracorporeal shock wave therapy is utilized to augment regenerative activity of the cells. In some embodiments, mesenchymal stem cells are administered. In some embodiments bone marrow cells are utilized as a source of mesenchymal stem cells. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, adipose-derived stem and regenerative cells (ADRCs). In one embodiment MSC donor lots are generated from umbilical cord tissue. Means of generating umbilical cord tissue MSC have been previously published and are incorporated by reference [53-59]. The term “umbilical tissue derived cells (UTC)” refers, for example, to cells as described in U.S. Pat. Nos. 7,510,873, 7,413,734, 7,524,489, and 7,560,276.

In one embodiment of the invention, granulocyte colony stimulating factor is utilized to mobilize stem cells, after which ultrasound waves are administered to the penile area. In other embodiments, bone marrow cells are administered. Granulocyte colony stimulating factor (G-CSF) has been used clinically for mobilization of hematopoietic stem cells (HSC) for more than a decade during donor stem cell harvesting. Mechanistically G-CSF is believed to induce a MMP-dependent alteration of the SDF-1 gradient in the bone marrow [60, 61], as well as function through a complement-dependent remodeling of the bone marrow extracellular matrix [62, 63]. It was found that in addition to mobilizing HSC, G-CSF stimulates mobilization of EPC as well, through mechanisms that are believed to be related [64, 65]. Several studies have been performed in which G-CSF was administered subsequent to infarct. Although it is impossible to state whether the mobilization of HSC or EPC accounted for the beneficial effects, we will overview some of these studies. The Front-Integrated Revascularization and Stem Cell Liberation in Evolving Acute Myocardial Infarction by Granulocyte Colony-Stimulating Factor (FIRSTLINE-AMI) trial evaluated 30 patients with ST-elevation myocardial infarction treated with control or G-CSF after successful revascularization [66]. Fifteen patients received 6 days of G-CSF at 10 μg/kg body weight, whereas the other 15 received standard care only. Four months after the infarct, the group that received G-CSF possessed a thicker myocardial wall at the area of infarct, as compared to controls. This was sustained over a year. Statistically significant improvements in ejection fraction, as well as inhibition of pathological remodeling was observed in comparison to controls. A larger subsequent study with 114 patients, 56 treated and 58 control demonstrated “no influence on infarct size, left ventricular function, or coronary restenosis” [67]. There may be a variety of reasons to explain the discrepancy between the trials. One most obvious one is that the mobilization was conducted immediately after the heart attack, whereas it may be more beneficial to time the mobilization with the timing of the chemotactic gradient released by the injured myocardium. This has been used to explain discrepancies between similar regenerative medicine trials [68]. Supporting this possibility is a study in which altered dosing was used for the successful improvement in angina [69]. Furthermore, a recent study last year demonstrated that in 41 patients with large anterior wall AMI an improvement in lef ventricle ejection fraction (LVEF) and diminished pathological remodeling was observed [70]. There is an indication that post-infarct mobilization can have a therapeutic role. Other clinically-applicable mobilizers may be evaluated and used in the methods described herein. For example, growth hormone, which is used in “antiaging medicine” has been demonstrated to improve endothelial responsiveness in healthy volunteers [71], and patients with congestive heart failure [72], this appears to be mediated through mobilization of endothelial progenitor cells [73, 74]. Thus in one embodiment of the present technology, the “optimizing” of EPC levels is performed so as to allow for enhanced cells numbers that are focused to the area of need. For example, interventions can include continuing with a course of treatment, discontinuing a course of treatment, increasing or decreasing the frequency or dosage levels of one or more treatments.

Within the practice of the invention, varying types of waveforms may be used to enhance stem cell activity. In some embodiments, the stimulation waveform comprises a series of frequency modulated pulses. The stimulation waveform can comprise a component selected from the group consisting of: a signal that modulates between 2.0 kHz and 3.0 kHz every second; a carrier frequency between 1 kHz and 50 kHz; a modulation frequency between 0.1 Hz and 10 kHz; a modulation range between 1 Hz and the carrier frequency; and combinations thereof. The stimulation waveform can comprise a pulse shape selected from the group consisting of: rectangle; triangle; symmetric triangle; asymmetric triangle; trapezoid; sawtooth; ramp; and combinations thereof. The stimulation waveform can comprise an amplitude that is varied. The amplitude can be modulated independent of the frequency modulation. In some embodiments, the stimulation waveform comprises pulses modulated by a wave selected from the group consisting of: rectangle wave; triangle wave; symmetric triangle wave; asymmetric triangle wave; trapezoid wave; sawtooth wave; ramp wave; and combinations thereof. The stimulation waveform can comprise a signal with a frequency greater than 1 kHz. The stimulation waveform can comprise pulses with a pulse width between 1 μs and 10 msec. The stimulation waveform can comprise pulses with a pulse width between 10 μs and 300 μs. The stimulation waveform can comprise cathodic pulses and anodic pulses. The stimulation waveform can be charge balanced. In some embodiments, the at least one implantable functional element comprises a first functional element and a second functional element, and the first functional element is configured to deliver stimulation energy at a first frequency, and the second functional element is configured to deliver stimulation energy at a second frequency different than the first frequency. The first frequency can be a high frequency and the second frequency can be a low frequency. The first functional element and the second functional element can deliver stimulation energy simultaneously. In some embodiments, the stimulation waveform comprises a first burst at a first frequency and a first amplitude, a second burst at a second frequency and a second amplitude, and at least one of: the first frequency and the second frequency are different or the first amplitude and the second amplitude are different. In some embodiments, the medical apparatus further comprises a first external device, and the first implantable device receives a power transmission from the first external device, and the stimulation waveform is independent of the power transmission. In some embodiments, the stimulation waveform comprises a current amplitude between 0.01 mA and 15 mA, such as between 0.1 mA and 15 mA. The stimulation waveform can comprise a current amplitude between 0.1 mA and 12 mA. The stimulation waveform can comprise a current amplitude between 0.1 mA and 10 mA. In some embodiments, the stimulation waveform comprises a burst. The burst can comprise low frequency signals. The burst can comprise high frequency signals. The burst can comprise pulses with a pulse width between 5 μs and 1 msec. The burst can comprise a stimulation parameter that is randomly varied. The randomly varied stimulation parameter can comprise a parameter selected from the group consisting of: amplitude; average amplitude; peak amplitude; frequency; average frequency; period; phase; polarity; pulse shape; a duty cycle parameter; inter-pulse gap; polarity; burst-on period; burst-off period; inter-burst period; pulse train; train-on period; train-off period; inter-train period; drive impedance; duration of pulse and/or amplitude level; duration of stimulation waveform; repetition of stimulation waveform; an amplitude modulation parameter; a frequency modulation parameter; a burst parameter; a power spectral density parameter; an anode/cathode configuration parameter; amount of energy and/or power to be delivered; rate of energy and/or power delivery; time of energy delivery initiation; method of charge recovery; and combinations thereof. The burst can comprise pulses with a pulse shape selected from the group consisting of: sinusoid; square, rectangle; triangle; symmetric triangle; asymmetric triangle; trapezoid; sawtooth; ramp; linear ramp; exponential curve; piece-wise step function; and combinations thereof. The burst can comprise an inter-pulse gap with a first duration and an inter-train period with a second duration, and the first duration can be less than the second duration. The burst can comprise an inter-pulse gap with a first duration and an inter-train period with a second duration, and the first duration can comprise a time between 0.1 μs and the second duration. The burst can comprise an inter-pulse gap with a duration between 1 μs and 1 second. The burst can comprise an inter-train period with a duration between 1 μs and 24 hours. The burst can comprise an inter-burst period with a duration between 20 μs and 24 hours. The burst can comprise an inter-train period with a duration between 1 μs and 24 hours. The burst can comprise a train-on period between 10 μs and 24 hours. The burst can comprise a train envelope and/or burst envelope with a shape selected from the group consisting of: cosine; cosine-squared; sine; square; rectangle; triangle; symmetric triangle; asymmetric triangle; trapezoid: sawtooth; ramp; linear ramp; and combinations thereof. The burst can comprise a train ramp duration and/or burst ramp duration between 1 μs and 10 minutes. The burst can comprise a depth of modulation between 1% and 99%. The burst can comprise pulses with a shape selected from the group consisting of: sinusoid; square; rectangle; triangle; symmetric triangle; asymmetric triangle; trapezoid; sawtooth; ramp; and combinations thereof. The burst can comprise a repeated pulse train comprising a stimulation parameter selected from the group consisting of: 3 to 6 pulses in each train; pulse widths of approximately 1 msec; inter-pulse gap of approximately 4 msec; inter-train period of approximately 25 msec; a train-on period of approximately 16 msec; and combinations thereof. The burst can comprise pulses with a pulse width between 5 μs and 1 msec. The burst can comprise an inter-pulse gap with a duration between 20 μs and 1 sec. The inter-pulse gap can vary with a variation magnitude between 0.1 μs and 1 msec. The burst can comprise an inter-burst period with a duration between 20 μs and 24 hours. The inter-burst period can vary with a variation magnitude between 1 μs and 24 hours. The burst can comprise a shaped burst envelope. The burst envelope can comprise a shape selected from the group consisting of: cosine; cosine-squared; sine; trapezoid; ramp; square; rectangular; triangular; and combinations thereof. The burst can comprise a burst ramp duration between 1 μs and 10 minutes.

### Example 1

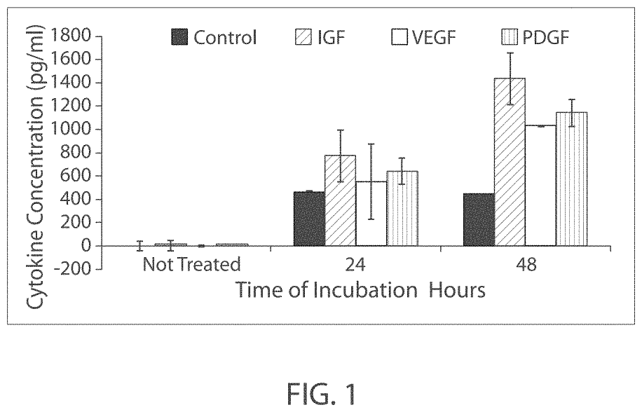

Bone marrow mononuclear cells where purchased from AllCells, Inc. and resuspended in RPMI media containing 10% fetal calf serum and 0.1% antibiotic/antimycotic after thawing and washing twice in phosphate buffered saline. 4.5-cm-long conical tubes (Thermo Fisher Scientific, Waltham, Mass., USA) at 1.0×105/mL were used. Extracorporeal shock waves where administered using a Duolith SD-1® device (StorzMedical, Tagerwilen, Switzerland) with an electromagnetic cylindrical coil source of focused shock wave. Cells were treated with 1000 impulses/cm2 at 0.1 mJ/mm2 of energy flux density, with a frequency of 4 Hz. After ESWT, the bone marrow mononuclear cells were maintained for 24 hr or 48 hrs. A control culture of cells not treated with shock waves was performed under similar conditions (24 hours) culture. Production of regenerative cytokines: IGF, VEGF, and PDGR was quantified by ELISA and the results are displayed in FIG. 1.

### REFERENCES

1. Kim, Y., et al., Injection of skeletal muscle-derived cells into the penis improves erectile function. Int J Impot Res, 2006. 18(4): p. 329-34.
2. Chancellor, M. B., et al., Gene therapy strategies for urological dysfunction. Trends Mol Med, 2001. 7(7): p. 301-6.
3. Christ, G. J., et al., Intracorporal injection of hSlo cDNA in rats produces physiologically relevant alterations in penile function. Am J Physiol, 1998. 275(2 Pt 2): p. H600-8.
4. Bivalacqua, T. J. and W. J. Hellstrom, Potential application of gene therapy for the treatment of erectile dysfunction. J Androl, 2001. 22(2): p. 183-90.
5. Mills, J. N., et al., Gene therapy for erectile dysfunction. Pharmacogenomics, 2007. 8(8): p. 979-84.
6. Melman, A., Gene therapy for male erectile dysfunction. Urol Clin North Am, 2007. 34(4): p. 619-30, viii.
7. Champion, H. C., et al., Gene transfer of endothelial nitric oxide synthase to the penis augments erectile responses in the aged rat. Proc Natl Acad Sci USA, 1999. 96(20): p. 11648-52.
8. Bivalacqua, T. J., et al., Adenoviral gene transfer of endothelial nitric oxide synthase (eNOS) to the penis improves age-related erectile dysfunction in the rat. Int J Impot Res, 2000. 12 Suppl 3: p. S8-17.
9. Tirney, S., et al., Nitric oxide synthase gene therapy for erectile dysfunction: comparison of plasmid, adenovirus, and adenovirus-transduced myoblast vectors. Mol Urol, 2001. 5(1): p. 37-43.
10. Magee, T. R., et al., Gene therapy of erectile dysfunction in the rat with penile neuronal nitric oxide synthase. Biol Reprod, 2002. 67(1): p. 20-8.
11. Magee, T. R., et al., Gene therapy of erectile dysfunction in the rat with penile neuronal nitric oxide synthase. Biol Reprod, 2002. 67(3): p. 1033-41.
12. Bivalacqua, T. J., et al., Gene transfer of endothelial nitric oxide synthase partially restores nitric oxide synthesis and erectile function in streptozotocin diabetic rats. J Urol, 2003. 169(5): p. 1911-7.
13. Chancellor, M. B., et al., Nitric oxide synthase gene transfer for erectile dysfunction in a rat model. BJU Int, 2003. 91(7): p. 691-6.
14. Bivalacqua, T. J., et al., Gene therapy techniques for the delivery of endothelial nitric oxide synthase to the corpora cavernosa for erectile dysfunction. Methods Mol Biol, 2004. 279: p. 173-85.
15. Bivalacqua, T. J., et al., Endothelial nitric oxide synthase gene therapy for erectile dysfunction. Curr Pharm Des, 2005. 11(31): p. 4059-67.
16. Christ, G. J., et al., Ion Channels and Gap Junctions: Their Role in Erectile Physiology, Dysfunction, and Future Therapy. Mol Urol, 1999. 3(2): p. 61-73.
17. Christ, G. J., s. J Androl, 2002. 23(5): p. S10-9.
18. Archer, S. L., Potassium channels and erectile dysfunction. Vascul Pharmacol, 2002. 38(1): p. 61-71.
19. Melman, A., et al., The successful long-term treatment of age related erectile dysfunction with hSlo cDNA in rats in vivo. J Urol, 2003. 170(1): p. 285-90.
20. Schiff, J. D. and A. Melman, Ion channel gene therapy for smooth muscle disorders: relaxing smooth muscles to treat erectile dysfunction. Assay Drug Dev Technol, 2006. 4(1): p. 89-95.
21. So, I., M. R. Chae, and S. W. Lee, Gene transfer of the K(ATP) channel restores age-related erectile dysfunction in rats. BJU Int, 2007. 100(5): p. 1154-60.
22. Bivalacqua, T. J., et al., Gene transfer of prepro-calcitonin gene-related peptide restores erectile function in the aged rat. Biol Reprod, 2001. 65(5): p. 1371-7.
23. Rogers, R. S., et al., Intracavernosal vascular endothelial growth factor (VEGF) injection and adeno-associated virus-mediated VEGF gene therapy prevent and reverse venogenic erectile dysfunction in rats. Int J Impot Res, 2003. 15(1): p. 26-37.
24. Seftel, A., Intracavernosal vascular endothelial growth factor (VEGF) injection and adeno-associated virus-mediated VEGF gene therapy and reverse venogenic erectile dysfunction in rats. J Urol, 2003. 170(2 Pt 1): p. 681.
25. Burchardt, M., et al., Application of angiogenic factors for therapy of erectile dysfunction: protein and DNA transfer of VEGF 165 into the rat penis. Urology, 2005. 66(3): p. 665-70.
26. Dall′Era, J. E., et al., Vascular endothelial growth factor (VEGF) gene therapy using a nonviral gene delivery system improves erectile function in a diabetic rat model. Int J Impot Res, 2008. 20(3): p. 307-14.
27. Qiu, X., et al., Combined strategy of mesenchymal stem cell injection with vascular endothelial growth factor gene therapy for the treatment of diabetes-associated erectile dysfunction. J Androl, 2012. 33(1): p. 37-44.
28. Liu, G., et al., Correction of diabetic erectile dysfunction with adipose derived stem cells modified with the vascular endothelial growth factor gene in a rodent diabetic model. PLoS One, 2013. 8(8): p. e72790.
29. Gholami, S. S., et al., The effect of vascular endothelial growth factor and adeno-associated virus mediated brain derived neurotrophic factor on neurogenic and vasculogenic erectile dysfunction induced by hyperlipidemia. J Urol, 2003. 169(4): p. 1577-81.
30. Wang, B., et al., Low-Intensity Extracorporeal Shock Wave Therapy Enhances Brain-Derived Neurotrophic Factor Expression through PERK/ATF4 Signaling Pathway. Int J Mol Sci, 2017. 18(2).
31. Shen, Z. J., et al., Gene transfer of vasoactive intestinal polypeptide into the penis improves erectile response in the diabetic rat. BJU Int, 2005. 95(6): p. 890-4.
32. Bennett, N. E., et al., Improvement in erectile dysfunction after neurotrophic factor gene therapy in diabetic rats. J Urol, 2005. 173(5): p. 1820-4.
33. Ryu, J. K., et al., Combined angiopoietin-1 and vascular endothelial growth factor gene transfer restores cavernous angiogenesis and erectile function in a rat model of hypercholesterolemia. Mol Ther, 2006. 13(4): p. 705-15.
34. Bivalacqua, T. J., et al., Superoxide anion production in the rat penis impairs erectile function in diabetes: influence of in vivo extracellular superoxide dismutase gene therapy. J Sex Med, 2005. 2(2): p. 187-97; discussion 197-8.
35. Pu, X. Y., et al., Improvement in erectile dysfunction after insulin-like growth factor-1 gene therapy in diabetic rats. Asian J Androl, 2007. 9(1): p. 83-91.
36. Magee, T. R., et al., Antisense and short hairpin RNA (shRNA) constructs targeting PIN (Protein Inhibitor of NOS) ameliorate aging-related erectile dysfunction in the rat. J Sex Med, 2007. 4(3): p. 633-643.
37. Kato, R., et al., Herpes simplex virus vector-mediated delivery of glial cell line-derived neurotrophic factor rescues erectile dysfunction following cavernous nerve injury. Gene Ther, 2007. 14(18): p. 1344-52.
38. Kato, R., et al., Herpes simplex virus vector-mediated delivery of neurturin rescues erectile dysfunction of cavernous nerve injury. Gene Ther, 2009. 16(1): p. 26-33.
39. Wu, X., et al., Human telomerase reverse transcriptase transfection reduces apoptosis in human penile smooth muscle cells and slows down cellular aging. J Sex Med, 2012. 9(2): p. 494-504.
40. Ryu, J. K., et al., Gene therapy with an erythropoietin enhancer-mediated, hypoxia-inducible gene expression system in the corpus cavernosum of mice with high-cholesterol diet-induced erectile dysfunction. J Androl, 2012. 33(5): p. 845-53.
41. Yang, Z., et al., Short hairpin ribonucleic acid constructs targeting insulin-like growth factor binding protein-3 ameliorates diabetes mellitus-related erectile dysfunction in rats. Urology, 2013. 81(2): p. 464 ell-6.
42. Pu, X. Y., et al., shRNA constructs targeting IGFBP-3 alleviate age related erectile dysfunction in the rat. J Urol, 2014. 192(3): p. 990-6.
43. He, S., et al., Myocardin restores erectile function in diabetic rats: phenotypic modulation of corpus cavernosum smooth muscle cells. Andrologia, 2015. 47(3): p. 303-9.
44. Yin, G. N., et al., Inhibition of Ninjurin 1 restores erectile function through dual angiogenic and neurotrophic effects in the diabetic mouse. Proc Natl Acad Sci USA, 2014. 111(26): p. E2731-40.
45. Zhu, X., et al., Improving erectile function of spontaneously hypertensive rats by silencing ROCK2. Urology, 2014. 84(4): p. 983 ell-8.
46. Tang, Z., et al., Human tissue kallikrein 1 ameliorates erectile function via modulation of macroautophagy in aged transgenic rats. Andrology, 2018. 6(5): p. 766-774.
47. Melman, A., et al., The first human trial for gene transfer therapy for the treatment of erectile dysfunction: preliminary results. Eur Urol, 2005. 48(2): p. 314-8.
48. Melman, A., et al., hMaxi-K gene transfer in males with erectile dysfunction: results of the first human trial. Hum Gene Ther, 2006. 17(12): p. 1165-76.
49. Melman, A., et al., Plasmid-based gene transfer for treatment of erectile dysfunction and overactive bladder: results of a phase I trial. Isr Med Assoc J, 2007. 9(3): p. 143-6.
50. Carneiro, F. S., et al., TNF-alpha infusion impairs corpora cavernosa reactivity. J Sex Med, 2009. 6 Suppl 3: p. 311-9.
51. Demirtas Sahin, T., et al., TNF-alpha antagonism with etanercept enhances penile NOS expression, cavernosal reactivity, and testosterone levels in aged rats. Can J Physiol Pharmacol, 2018. 96(2): p. 200-207.
52. Demirtas Sahin, T., et al., Depression induced by chronic stress leads to penile cavernosal dysfunction: protective effect of anti-TNF-alpha treatment. Can J Physiol Pharmacol, 2018. 96(9): p. 933-942.
53. Van Pham, P., et al., Isolation and proliferation of umbilical cord tissue derived mesenchymal stem cells for clinical applications. Cell Tissue Bank, 2015.
54. Fazzina, R., et al., A new standardized clinical-grade protocol for banking human umbilical cord tissue cells. Transfusion, 2015. 55(12): p. 2864-73.
55. Bieback, K., Platelet lysate as replacement for fetal bovine serum in mesenchymal stromal cell cultures. Transfus Med Hemother, 2013. 40(5): p. 326-35.
56. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.
57. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.
58. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.
59. Friedman, R., et al., Umbilical cord mesenchymal stem cells: adjuvants for human cell transplantation. Biol Blood Marrow Transplant, 2007. 13(12): p. 1477-86.
60. Jin, F., et al., Degradation of BM SDF-1 by MMP-9: the role in G-CSF-induced hematopoietic stem/progenitor cell mobilization. Bone Marrow Transplant, 2008. 42(9): p. 581-8.
61. Carion, A., et al., Stromal-derived factor 1 and matrix metalloproteinase 9 levels in bone marrow and peripheral blood of patients mobilized by granulocyte colony-stimulating factor and chemotherapy. Relationship with mobilizing capacity of haematopoietic progenitor cells. Br J Haematol, 2003. 122(6): p. 918-26.
62. Ratajczak, M. Z., et al., A pivotal role of activation of complement cascade (CC) in mobilization of hematopoietic stem/progenitor cells (HSPC). Adv Exp Med Biol, 2008. 632: p. 47-60.
63. Lee, H. M., et al., Impaired mobilization of hematopoietic stem/progenitor cells in C5-deficient mice supports the pivotal involvement of innate immunity in this process and reveals novel promobilization effects of granulocytes. Leukemia, 2009.
64. Pitchford, S. C., et al., Differential mobilization of subsets of progenitor cells from the bone marrow. Cell Stem Cell, 2009. 4(1): p. 62-72.
65. Korbling, M., et al., Recombinant human granulocyte-colony-stimulating factor-mobilized and apheresis-collected endothelial progenitor cells: a novel blood cell component for therapeutic vasculogenesis. Transfusion, 2006. 46(10): p. 1795-802.
66. Ince, H., et al., Prevention of left ventricular remodeling with granulocyte colony-stimulating factor after acute myocardial infarction: final 1-year results of the Front-Integrated Revascularization and Stem Cell Liberation in Evolving Acute Myocardial Infarction by Granulocyte Colony-Stimulating Factor (FIRSTLINE-AMI) Trial. Circulation, 2005. 112(9 Suppl): p. 173-80.
67. Zohlnhofer, D., et al., Stem cell mobilization by granulocyte colony-stimulating factor in patients with acute myocardial infarction: a randomized controlled trial. JAMA, 2006. 295(9): p. 1003-10.
68. Hill, J. M. and J. Bartunek, The end of granulocyte colony-stimulating factor in acute myocardial infarction? Reaping the benefits beyond cytokine mobilization. Circulation, 2006. 113(16): p. 1926-8.
69. Suzuki, K., et al., Effect of granulocyte colony-stimulating factor treatment at a low dose but for a long duration in patients with coronary heart disease. Circ J, 2006. 70(4): p. 430-7.
70. Leone, A. M., et al., Usefulness of granulocyte colony-stimulating factor in patients with a large anterior wall acute myocardial infarction to prevent left ventricular remodeling (the rigenera study). Am J Cardiol, 2007. 100(3): p. 397-403.
71. Napoli, R., et al., Acute effects of growth hormone on vascular function in human subjects. J Clin Endocrinol Metab, 2003. 88(6): p. 2817-20.
72. Napoli, R., et al., Growth hormone corrects vascular dysfunction in patients with chronic heart failure. J Am Coll Cardiol, 2002. 39(1): p. 90-5.
73. Devin, J. K., et al., Low-dose growth hormone administration mobilizes endothelial progenitor cells in healthy adults. Growth Horm IGF Res, 2008. 18(3): p. 253-63.
74. Thum, T., et al., Age-dependent impairment of endothelial progenitor cells is corrected by growth-hormone-mediated increase of insulin-like growth-factor-1. Circ Res, 2007. 100(3): p. 434-43.

1. A method for the stimulation of penile regeneration, the method comprising: a) associating the corpus cavernosum of a patient in need of therapy with a shockwave generating device; b) applying a shockwave regimen to said corpus cavernosum wherein said shockwave device produces a focal zone comprising at least a portion of the corpus cavernosum; and c) administering a regenerative cell population.

2. The method of claim 1 wherein said shockwave regimen locally promotes at least one or more biological activities chosen from the group consisting of: a) angiogenesis; b) enhanced perfusion of the penis, c) mitogenesis of smooth muscle cells; and d) augmentation of endothelial function.

3. The method of claim 1 wherein said shock wave regimen produce a treatment regimen determined based on at least one parameter chosen from the group consisting of shockwave parameters, treatment protocol parameters, and anatomical parameters.

4. The method of claim 3 wherein said shockwave parameters comprise number of shockwaves, frequency of shockwaves and intensity of said shockwave.

5. The method of claim 4 wherein said parameters are selected from of at least one of: shockwave intensity is about from about 50 bar to about 200 bar; shockwave frequency is from about 60 to about 300 shockwaves per min; said number of shockwaves is up to about 3500 per session.

6. The method of claim 1 further comprising coupling said shock wave regimen with a drug or cellular treatment.

7. The method of claim 1, wherein an additional therapeutic agent is administered into the corpus cavernosum.

8. The method of claim 7, wherein the additional therapeutic agent is selected from the group consisting of growth factors, differentiation factors, regenerative cells, and nutritional supplements.

9. The method of claim 8, wherein the additional therapeutic agent is a growth factor.

10. The method of claim 9, wherein the additional therapeutic agent and the cells are administered into the corpus cavernosum using a carrier.

11. The method of claim 10, wherein the carrier is selected from the group consisting of beads, microspheres, nanospheres, hydrogels, gels, polymers, ceramics, collagen and platelet gels.

12. The method of claim 9, wherein the additional therapeutic agent is administered simultaneously with administering the cells to the corpus cavernosum.

13. The method of claim 9, wherein the additional therapeutic agent is administered prior to administering the cells to the corpus cavernosum.

14. The method of claim 9, wherein the additional therapeutic agent is administered after administering the cells to the corpus cavernosum.

15. The method of claim 9, wherein the cells are administered into the corpus cavernosum in a formulation with a volume of between about 0.1 ml and about 2 ml.

16. A method of augmenting therapeutic activity of bone marrow cells comprising exposing said bone marrow cells to extracorporeal shock waves.

17. The method of claim 16, wherein said extracorporeal shock waves possess parameters are selected from of at least one of: shockwave intensity is about from about 50 bar to about 200 bar; shockwave frequency is from about 60 to about 300 shockwaves per min; said number of shockwaves is up to about 3500 per session.

18. A method of augmenting therapeutic activity of regenerative cells comprising exposing said bone marrow cells to extracorporeal shock waves.

19. The method of claim 18, wherein said extracorporeal shock waves possess parameters are selected from of at least one of: shockwave intensity is about from about 50 bar to about 200 bar; shockwave frequency is from about 60 to about 300 shockwaves per min; said number of shockwaves is up to about 3500 per session.

20. The method of claim 19, wherein said regenerative cells are mesenchymal stem cells.
